SEQUENCE LISTING (10 sequences)

SEQ ID NO: 1 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - DNA, 855 bp, Artificial Sequence
  Synthetic polynucleotide
atgcggtacacagggctaatgggaacactgctcaccttggtcaacctgctgcagctggctgggactctgagaattgcagccttcaacattcggacttttggggagactaagatgtccaatgctaccctctctgtatactttgtgaaaatcctgagtcgctatgacatcgctgttatccaagaggtcagagactcccacctggttgctgttgggaagctcctggatgaactcaatcgggacaaacctgacacctaccgctatgtagtcagtgagccgctgggccgcaaaagctacaaggaacagtacctttttgtgtacaggcctgaccaggtgtctattctggacagctatcaatatgatgatggctgtgaaccctgtggaaatgacaccttcagcagagagccagccattgttaagttcttttccccatacactgaggtccaagaatttgcgatcgtgcccttgcatgcagccccaacagaagctgtgagtgagatcgacgccctctacgatgtttacctagatgtctggcaaaagtggggcctggaggacatcatgttcatgggagacttcaatgctggctgcagctacgtcacttcctcccagtggtcctccattcgccttcggacaagccccatcttccagtggctgatccctgacagtgcggacaccacagtcacatcaacacactgtgcttatgacaggattgtggttgctggagctctgctccaggctgctgttgttcccaactcggctgttccttttgatttccaagcagaatacggactttccaaccagctggctgaagccatcagtgaccattacccagtggaggtgacactcagaaaaatctga

SEQ ID NO: 3 - protein, 284 aa, Artificial Sequence
  Synthetic polypeptide
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKI

SEQ ID NO: 4 - DNA, 554 bp, Artificial Sequence
  Synthetic polynucleotide
tacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatc

SEQ ID NO: 5 - DNA, 2236 bp, Artificial Sequence
  Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatgcggtacacagggctaatgggaacactgctcaccttggtcaacctgctgcagctggctgggactctgagaattgcagccttcaacattcggacttttggggagactaagatgtccaatgctaccctctctgtatactttgtgaaaatcctgagtcgctatgacatcgctgttatccaagaggtcagagactcccacctggttgctgttgggaagctcctggatgaactcaatcgggacaaacctgacacctaccgctatgtagtcagtgagccgctgggccgcaaaagctacaaggaacagtacctttttgtgtacaggcctgaccaggtgtctattctggacagctatcaatatgatgatggctgtgaaccctgtggaaatgacaccttcagcagagagccagccattgttaagttcttttccccatacactgaggtccaagaatttgcgatcgtgcccttgcatgcagccccaacagaagctgtgagtgagatcgacgccctctacgatgtttacctagatgtctggcaaaagtggggcctggaggacatcatgttcatgggagacttcaatgctggctgcagctacgtcacttcctcccagtggtcctccattcgccttcggacaagccccatcttccagtggctgatccctgacagtgcggacaccacagtcacatcaacacactgtgcttatgacaggattgtggttgctggagctctgctccaggctgctgttgttcccaactcggctgttccttttgatttccaagcagaatacggactttccaaccagctggctgaagccatcagtgaccattacccagtggaggtgacactcagaaaaatctgaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 21 bp, Artificial Sequence
  Synthetic polynucleotide
tcgctatgacatcgctgttat

SEQ ID NO: 7 - DNA, 21 bp, Artificial Sequence
  Synthetic polynucleotide
gtcaggtttgtcccgattgag

SEQ ID NO: 8 - DNA, 22 bp, Artificial sequence
  Synthetic polynucleotide
tggcaaagtggagattgttgcc

SEQ ID NO: 9 - DNA, 21 bp, Artificial sequence
  Synthetic polynucleotide
aagatggtgatgggcttcccg

SEQ ID NO: 10 - protein, 282 aa, Homo sapiens [sequence omitted]

(19) United States
(12) Patent Application Publication
(10) Pub. No.: US 2018/0214576 A1
(43) Pub. Date: Aug. 2, 2018

(54) TRANSGENIC EXPRESSION OF DNASEI IN VIVO DELIVERED BY AN ADENO-ASSOCIATED VIRUS VECTOR

(71) Applicant: University of Massachusetts, Boston, MA (US)

(72) Inventors: Katherine A. Fitzgerald, Wellesley, MA (US); Stefan A. Schattgen, Memphis, TN (US); Guangping Gao, Westborough, MA (US)

(73) Assignee: University of Massachusetts, Boston, MA (US)

(21) Appl. No.: 15/747,801

(22) PCT Filed: Jul. 28, 2016
(86) PCT No.: PCT/US16/44512

§ 371 Date: Jan. 26, 2018

Related U.S. Application Data
(60) Provisional application No. 62/198,100, filed on Jul. 28, 2015.

(51) Int. Cl. A61K 48/00; C12N 15/63; C12N 9/22; A61K 9/00; A61P 37/02

(52) U.S. Cl. CPC A61K 48/0058; C12N 15/63; A61P 37/02; A61K 9/007; C12N 9/22

(57) ABSTRACT

The disclosure relates to compositions and methods for AAV-mediated delivery of a nuclease to a subject. In some embodiments, the nuclease is DNAse I. In some embodiments, the methods are useful for treatment of lung-associated diseases.

### RELATED APPLICATIONS

This application is a National Stage Application of PCT/US2016/044512, filed Jul. 28, 2016 entitled “TRANSGENIC EXPRESSION OF DNASE I IN VIVO DELIVERED BY AN ADENO-ASSOCIATED VIRUS VECTOR”, which claims the benefit under 35 U.S.C. § 119(e) of U.S. provisional application 62/198,100, filed Jul. 28, 2015, entitled “TRANSGENIC EXPRESSION OF DNASE I IN VIVO DELIVERED BY AN ADENO-ASSOCIATED VIRUS VECTOR”, the entire contents of each application which are incorporated herein by reference.

### FEDERALLY SPONSORED RESEARCH

This invention was made with government support under AI093752, AI083713, AI095213, awarded by the National Institutes of Health. The government has certain rights in the invention.

### BACKGROUND OF INVENTION

Humans carry a gene encoding for DNase I, which is translated into a secreted enzyme that enters the extracellular space and bloodstream. DNase I specifically degrades long deoxyribonucleic acid (DNA) polymers into nucleotide monomers. DNA constitutes a major component of mucus in cystic fibrosis (CF) patients and contributes to its viscosity. Clogging of the airways of CF patients is a result of mucus build-up due to defective mucocilliary clearance. Recombinant DNase I has been used to address this build-up by degrading DNA in the mucus of CF patients. The viscosity of sputum in CF patients can be reduced in a dose-dependent manner following treatment with recombinant DNase I.

### SUMMARY OF INVENTION

Aspects of the disclosure relate to methods and compositions for delivering recombinant nucleases to a cell or tissue, e.g., for treating lung conditions. Conventionally, CF patients have been treated with recombinant DNase I delivered via nebulizer twice daily. There are several drawbacks to this delivery mechanism and treatment regimen, for example short and limited DNase I bioavailability, patient adherence, and production cost of DNase I. Recombinant DNase I must be glycosylated following translation in order to gain full activity and thermal stability. This increases production costs and complexity for recombinant DNase I and generally precludes the use of less costly prokaryotic overexpression systems. Accordingly, the inventors have developed new compositions and methods related to the delivery of recombinant nucleases.

Aspects of the disclosure relate to compositions and methods for recombinant adeno-associated virus (AAV)-mediated delivery of a transgene encoding a nuclease (e.g., DNase I).

In some aspects, the disclosure relates to a recombinant AAV (rAAV) comprising: a capsid protein having a sequence as set forth in SEQ ID NO: 1 and a nucleic acid comprising a promoter operably linked to a transgene, wherein the transgene encodes a nuclease.

In some embodiments, the nuclease is DNase I. In some embodiments, the DNase I is human DNase I (e.g., GenBank ID NM_005223.3 or GenBank ID NP_005214.2) or mouse DNase I (e.g., GenBank ID NM_010061.5 or GenBank ID NP_034191.3). In some embodiments, the transgene comprises a sequence as set forth in SEQ ID NO: 2 or encodes a protein as set forth in SEQ ID NO: 3 or 10.

In some embodiments, the nucleic acid further comprises two AAV inverted terminal repeats (ITRs), wherein the ITRs flank the transgene. In some embodiments, the AAV ITRs are AAV2, AAV3, AAV4, AAV5, or AAV6 ITRs.

In some embodiments, the promoter comprises a sequence as set forth in SEQ ID NO: 4.

In some aspects, the disclosure relates to a composition comprising an rAAV and a pharmaceutically acceptable carrier.

In some aspects, the disclosure relates to an isolated nucleic acid having the sequence as set forth in SEQ ID NO: 5.

In some aspects, the disclosure relates to a vector comprising the isolated nucleic acid of having the sequence as set forth in SEQ ID NO: 5.

In some aspects, the disclosure relates to a host cell comprising an isolated nucleic acid or vector as described by the disclosure. In some embodiments, the host cell is a human cell or bacterial cell.

In some aspects, the disclosure relates to a method for delivering a transgene to lung tissue, the method comprising: administering to lung tissue of a subject an effective amount of rAAV, wherein the rAAV comprises (i) a capsid protein and (ii) a nucleic acid comprising a promoter operably linked to a transgene, wherein the transgene encodes a nuclease.

In some embodiments, the nuclease is DNase I. In some embodiments, the DNase I is human DNase I (e.g., GenBank ID NM_005223.3 or GenBank ID NP_005214.2) or mouse DNase I (e.g., GenBank ID NM_010061.5 or GenBank ID NP_034191.3). In some embodiments, the transgene comprises a sequence as set forth in SEQ ID NO: 2 or encodes a protein as set forth in SEQ ID NO: 3 or 10.

In some embodiments, the capsid protein is selected from the group consisting of: AAV2, AAV3, AAV4, AAV5, AAV6, AAV7, AAV8, or AAV9 capsid protein. In some embodiments, the capsid protein comprises the sequence set forth in SEQ ID NO: 1.

In some embodiments, the nucleic acid further comprises two AAV inverted terminal repeats (ITRs), wherein the ITRs flank the transgene. In some embodiments, the AAV ITRs are AAV2, AAV3, AAV4, AAV5, or AAV6 ITRs.

In some embodiments, the promoter comprises a sequence as set forth in SEQ ID NO: 4.

In some embodiments, the administration is respiratory administration. In some embodiments, the respiratory administration comprises intranasal or intratracheal administration. In some embodiments, the respiratory administration comprises administration of aerosolized particles comprising the rAAV. In some embodiments, the respiratory administration is a self-administration. In some embodiments, the respiratory administration is a self-administration by inhalation.

In some aspects, the disclosure relates to a method for treating a lung-associated disease, the method comprising: administering to a subject having or suspected of having a lung-associated disease an effective amount of rAAV, wherein the rAAV comprises (i) a capsid protein and (ii) a nucleic acid comprising a promoter operably linked to a transgene, wherein the transgene encodes a nuclease.

In some embodiments, the treatment comprising administering a dose of the rAAV to the subject no more than once within a calendar day. In some embodiments, the treatment comprising administering a dose of the rAAV to the subject no more than once within a calendar week. In some embodiments, the treatment comprising administering a dose of the rAAV to the subject no more than once within a calendar month.

In some embodiments, the nuclease is DNase I. In some embodiments, the DNase I is human DNase I (e.g., GenBank ID NM_005223.3 or GenBank ID NP_005214.2) or mouse DNase I (e.g., GenBank ID NM_010061.5 or GenBank ID NP_034191.3). In some embodiments, the transgene comprises a sequence as set forth in SEQ ID NO: 3 or SEQ ID NO: 10.

In some embodiments, the capsid protein is selected from the group consisting of: AAV2, AAV3, AAV4, AAV5, AAV6, AAV7, AAV8, or AAV9 capsid protein. In some embodiments, the capsid protein comprises the sequence set forth in SEQ ID NO: 1.

In some embodiments, the nucleic acid further comprises two AAV inverted terminal repeats (ITRs), wherein the ITRs flank the transgene. In some embodiments, the AAV ITRs are AAV2, AAV3, AAV4, AAV5, AAV6 ITRs. In some embodiments, the promoter comprises a sequence as set forth in SEQ ID NO: 4.

In some embodiments, the administration is intranasal administration or respiratory administration.

In some embodiments, the lung-associated disease is selected from the group consisting of: cystic fibrosis (CF), asthma, chronic obstructive pulmonary disease (COPD), emphysema, bronchitis, pneumonia, tuberculosis, influenza, pulmonary edema, and acute respiratory distress syndrome (ARDS).

In some aspects, the disclosure relates to a method for inhibiting biofilm formation on a surface, the method comprising: administering to a surface an effective amount of rAAV, wherein the rAAV comprises (i) a capsid protein and (ii) a nucleic acid comprising a promoter operably linked to a transgene, wherein the transgene encodes a nuclease.

In some embodiments, the surface is a hard tissue or a soft tissue. In some embodiments, the surface is non-biological surface. In some embodiments, the surface is in vivo. In some embodiments, the surface is a surface of a biomedical implant.

In some aspects, the disclosure relates to a method for treating an autoimmune disease, the method comprising: administering to a subject having or suspected of having a lung-associated disease an effective amount of rAAV, wherein the rAAV comprises (i) a capsid protein and (ii) a nucleic acid comprising a promoter operably linked to a transgene, wherein the transgene encodes a nuclease.

In some embodiments, the autoimmune disease is systemic lupus erythematosus (SLE), polyarthritis, Aicardi-Goutieres Syndrome (AGS), or chilblain lupus.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF DRAWINGS

FIG. 1 shows a restriction map of pAAVsc CB6 PE murine DNase I which is 5040 nucleotides in length.

FIGS. 2A-2B illustrate DNA accumulating in the lung microenvironment during influenza A virus (IAV) infection. FIG. 2A shows H&E staining of lung tissue from uninfected and infected WT mice at 5 days post infection (dpi) at 100× magnification. Arrows indicate necrotic cells associated with hematoxylin-rich strands. Scale bar=10 μm FIG. 2B shows concentration of DNA in cell-depleted BAL fluid from PR8 infected WT mice at indicated times. DNA quantified by PicoGreen assay. *, P 0.05; **, P 0.01; ****, P 0.001.

FIGS. 3A-3H illustrate DNA in the lung is protective to IAV infection. FIG. 3A shows a schematic for transduced expression of DNase I. FIG. 3B shows dnase1 mRNA measured by qPCR with total RNA isolated from lungs of AAV-GFP and AAV-DNase I treated mice. FIG. 3C shows DNA in cell-depleted BAL fluid in uninfected controls compared to AAV-GFP and AAV-DNase I treated mice at 5 dpi with PR8. FIG. 3D shows a survival comparison between AAV-GFP and AAV-DNase I treated WT mice challenged with 4×10⁴ plaque forming units (pfu) PR8. Data combined from two independent experiments (AAV-GFP, n=17; AAV-DNaseI, n=15). FIG. 3E shows IAV viral RNA (vRNA) copy number measured by qPCR using total lung RNA of AAV-GFP and AAV-DNase I treated mice at 5 dpi with PR8. FIGS. 3F-3H show total number of CD45+ leukocytes (FIG. 3F), CD4+ T cells (FIG. 3G), and CD8+ T cells (FIG. 3H) in the total lungs of AAV-GFP and AAV-DNase I treated mice at 5 dpi with PR8. *, P 0.05.

FIGS. 4A-4G show AIM2 is protective during IAV infection. FIG. 4A shows a survival comparison of WT and Aim2^{-/-} mice infected with 4×10⁴ pfu PR8 (WT, n=6; Aim2^{-/-}, n=5). FIG. 4B shows a table illustrating the surviving proportion of WT and Aim2^{-/-} mice pooled from 3 independent experiments. FIG. 4C shows a survival comparison of lethally irradiated WT mice engrafted with WT and Aim2^{-/-} bone marrow infected with PR8 (WT>WT, n=10; Aim2⁻¹⁻>WT, n=9) FIG. 4D shows IAV vRNA copy number in the lung RNA from WT and Aim2^{-/-} at 3 and 5 dpi with PR8. FIGS. 4E-4G) Levels of IL-1β (FIG. 4E), IL-6 (FIG. 4F), and TNFα (FIG. 4G) protein in lung homogenates at 3 and 5 dpi as measured by ELISA.

FIGS. 5A-5G show AIM2 dampens the inflammatory response to IAV infection. FIG. 5A shows frequency of NP366-tetramer⁺ CD8⁺ T cells. FIG. 5B shows total number tetramer⁺ CD8⁺ T cells in the lungs of WT and Aim2^{-/-} mice at 9 dpi with 10³ pfu PR8 using three epitopes (NP366, PA224, PB703). Populations were gated on CD45⁺ CD44^{hi}CD8⁺ TCRβ⁺ cells. (FIGS. 5C-5G) Total number of CD45+ cells (FIG. 5C), CD4⁺ T cells (FIG. 5D), CD8⁺ T cells (FIG. 5E), CD11b⁺ dendritic cells (FIG. 5F), and immature macrophages (FIG. 5G) in the lung at 5 dpi with 4×10⁴ pfu PR8. *, P 0.05; **, P 0.01; ***, P 0.005.

FIG. 6 shows qPCR measurement of AAV-GFP and AAV-DNase I mRNA levels in lungs of mice three months after intranasal infection.

### DETAILED DESCRIPTION OF INVENTION

Aspects of the invention relate to the surprising discovery that delivery of nuclease enzymes (e.g., DNase I) using certain recombinant AAV-based gene transfer vectors is effective in reducing inflammatory immune responses due to release of nucleic acids (e.g., DNA) from damaged tissue. Accordingly, methods and compositions described by the disclosure are useful, in some embodiments, for the treatment of lung associated diseases (e.g., cystic fibrosis).

### Methods and Compositions for AAV-Mediated Delivery of Nucleases

Methods for delivering a transgene to lung tissue in a subject are provided herein. The methods typically involve administering to a subject an effective amount of a rAAV comprising a nucleic acid for expressing a transgene in the subject. In some embodiments, a transgene is a nuclease, for example DNase I. An “effective amount” of a rAAV is an amount sufficient to infect a sufficient number of cells of a target tissue in a subject. In some embodiments, a target tissue is lung tissue. An effective amount of a rAAV may be an amount sufficient to have a therapeutic benefit in a subject, e.g., to extend the lifespan of a subject, to improve in the subject one or more symptoms of disease, e.g., a symptom of cystic fibrosis (CF), a symptom of an autoimmune disease (for example, systemic lupus erythematosus (SLE), polyarthritis, or Aicardi-Goutières Syndrome), etc. In some cases, an effective amount of a rAAV may be an amount sufficient to produce a stable somatic transgenic animal model. The effective amount will depend on a variety of factors such as, for example, the species, age, weight, health of the subject, and the CNS tissue to be targeted, and may thus vary among subject and tissue.

An effective amount may also depend on the mode of administration. For example, targeting a lung tissue by intranasal administration may require different (e.g., higher) doses, in some cases, than targeting lung tissue by intratracheal injection or respiratory administration. In some cases, multiple doses of a rAAV are administered. An effective amount may also depend on the rAAV used. For example, dosages for targeting a lung tissue may depend on the serotype (e.g., the capsid protein) of the rAAV. For example, the rAAV may have a capsid protein of a AAV serotype selected from the group consisting of: AAV2, AAV3, AAV4, AAV5, AAV6, AAV7, or AAV9. In some embodiments, the rAAV comprises an AAV9 capsid protein. In some embodiments, the rAAV comprises a capsid protein having the sequence set forth in SEQ ID NO: 1. In certain embodiments, the effective amount of rAAV is 10¹⁰, 10¹¹, 10¹², 10¹³, or 10¹⁴ genome copies per kg. In certain embodiments, the effective amount of rAAV is 10¹⁰, 10¹¹, 10¹², 10¹³, 10¹⁴, or 10¹⁵ genome copies per subject.

Methods and compositions for treating lung-associated diseases and disorders are also provided herein. As used herein, a “lung-associated disorder” is a disease or condition of the lung. Non-limiting examples of lung-associated diseases include, but are not limited to, cystic fibrosis (CF), asthma, chronic obstructive pulmonary disease (COPD), emphysema, bronchitis, pneumonia, tuberculosis, influenza, pulmonary edema, and acute respiratory distress syndrome (ARDS). Often, lung-associated diseases are linked to inflammation of lung tissue caused by release of nucleic acids (e.g., DNA) by damaged tissue. Without wishing to be bound by any particular theory, rAAV-based delivery of a nuclease (e.g., DNase I) reduces inflammation of lung tissue in subjects having lung-associated disease.

The skilled artisan recognizes that other inflammatory conditions associated with the release of nucleic acids by damaged tissue may also be treated by methods and compositions described herein. For example, the accumulation of self-nucleic acids, including DNA, is linked to the development and progression of autoimmune disease. Examples of autoimmune disease include, but are not limited to, systemic lupus erythematosus (SLE), polyarthritis, Aicardi-Goutières Syndrome (AGS), and chilblain lupus. Accordingly, in some embodiments, the disclosure provides methods and compositions for the treatment of autoimmune disease.

In some aspects, the disclosure provides methods and compositions for inhibiting biofilm formation (e.g., biofilm growth). As used herein, the term “biofilm” refers to an aggregate of microorganisms (e.g., a colony of bacteria) that adhere to one another, thus forming a thin layer of cells that coat a surface. Generally, biofilms are formed when bacteria attach to surfaces and form structured multicellular communities. A biofilm can be formed by bacteria (e.g., gram-positive bacteria, gram-negative bacteria) or fungus (e.g., Candida albicans). The surface on which a biofilm forms can be an organic surface (e.g., biological surface) or an inorganic surface or non-biological surface (e.g., a plastic, a polymer, a metal). In some embodiments a surface is in vivo (e.g., situated inside a subject). For example, biofilm formation can occur in the context of a persistent disease (e.g., bacterial vaginosis) or in the context of contamination of a surgical instrument (e.g., a scalpel, forceps, etc.), a surgical device (e.g., a plate, a bone screw, or surgical wire), or a prosthesis (e.g., a prosthetic joint). A major component of biofilms contributing to their structural integrity is extracellular DNA. Without wishing to be bound by any particular theory, in some embodiments nucleases (e.g., DNase I) are effective in preventing the formation and disrupting preexisting biofilm formation.

Recombinant Adeno-Associated Viruses (rAAVs)

In some aspects, the disclosure provides isolated AAVs. As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV2, AAV3, AAV4, AAV5, AAV6, AAV8, AAVrh8, AAV9, and AAV10. In some embodiments, an AAV capsid protein is of a serotype derived from a non-human primate. In some embodiments, an AAV capsid protein is of a AAVrh8 serotype. In some embodiments, an AAV capsid protein is of an AAV9 serotype. In some embodiments, an AAV capsid protein is represented by the sequence set forth in SEQ ID NO: 1.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding a nuclease (e.g., DNase I). In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The foregoing methods for packaging recombinant vectors in desired AAV capsids to produce the rAAVs of the disclosure are not meant to be limiting and other suitable methods will be apparent to the skilled artisan.

### Isolated Nucleic Acids

A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

In some embodiments, an isolated nucleic acid encodes a nuclease. In some embodiments, an isolated nucleic acid encodes a transgene represented by the sequence set forth in SEQ ID NO: 3. In some embodiments, an isolated nucleic acid encodes a recombinant AAV vector (rAAV vector).

Recombinant AAV Vectors (rAAV Vectors)

“Recombinant AAV (rAAV) vectors” of the disclosure are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., gRNA) or other gene product, of interest. The nucleic acid coding sequence is operatively linked to regulatory components in a manner which permits transgene transcription, translation, and/or expression in a cell of a target tissue.

In some embodiments, the instant disclosure relates to a recombinant AAV (rAAV) vector comprising a nucleic acid sequence including a promoter operably linked to a transgene, wherein the transgene is a nuclease (e.g., DNase I). As used herein, the term “nuclease” refers to an enzyme that cleaves a phosphodiester bond or binds within a polynucleotide chain. Nucleases may be naturally occurring or genetically engineered. Examples of nucleases include, but are not limited to, DNAse I, DNAse II, DNAse IV, and restriction enzymes (e.g., NotI, Xba, etc.). In some embodiments, a rAAV vector further comprises nucleic acid sequences encoding one or more AAV inverted terminal repeat sequences (ITRs), for example AAV2 ITRs. In some embodiments, a rAAV vector comprises a nucleic acid having the sequence set forth in SEQ ID NO: 2.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types (e.g., AAV2, AAV3, AAV4, AAV5, or AAV6 ITR sequences).

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., gRNA).

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al, and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, Petal., Human Gene Therapy, 2000; 11: 1921-1931; and Klump, H et al., Gene Therapy, 2001; 8: 811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter, for example as represented by SEQ ID NO: 4.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, one or more bindings sites for one or more of miRNAs are incorporated in a transgene of a rAAV vector, to inhibit the expression of the transgene in one or more tissues of an subject harboring the transgene. The skilled artisan will appreciate that binding sites may be selected to control the expression of a transgene in a tissue specific manner. For example, binding sites for the liver-specific miR-122 may be incorporated into a transgene to inhibit expression of that transgene in the liver. The target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

### Recombinant AAV Administration Methods

The rAAVs may be delivered to a subject in compositions according to any appropriate methods known in the art. The rAAV, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the lung (or lungs) of a subject. By “lung” is meant all cells and tissue of the lung of a vertebrate. Thus, the term includes, but is not limited to, alveolar cells, pneumocytes, endothelial cells (e.g., capillary endothelial cells), alveolar macrophages, and the like. Recombinant AAVs may be delivered directly to the lung by respiratory administration (e.g., inhalation or delivery via nebulizer), intranasal administration, intratracheal administration, or any other suitable administration method.

Aspects of the instant disclosure relate to compositions comprising a recombinant AAV comprising a capsid protein and a nucleic acid encoding a nuclease (e.g., DNase I). In some embodiments, the nucleic acid further comprises AAV ITRs. In some embodiments, the rAAV comprises an rAAV vector represented by the sequence set forth in SEQ ID NO: 5. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target heart, liver, and pancreas tissues. In some cases, stable transgenic animals are produced by multiple doses of an rAAV.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered trangenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the instant disclosure relates to a kit for producing a rAAV, the kit comprising a container housing an isolated nucleic acid having a sequence of SEQ ID NO: 5. In some embodiments, the kit further comprises instructions for producing the rAAV. In some embodiments, the kit further comprises at least one container housing a recombinant AAV vector, wherein the recombinant AAV vector comprises a transgene (e.g., a nuclease, such as DNAse I).

In some embodiments, the instant disclosure relates to a kit comprising a container housing a recombinant AAV having an isolated AAV capsid protein having an amino acid sequence as set forth in any of SEQ ID NO: 1.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or iv needle tubing and bag, particularly in the case of the kits for producing specific somatic animal models.

In some cases, the methods involve transfecting cells with total cellular DNAs isolated from the tissues that potentially harbor proviral AAV genomes at very low abundance and supplementing with helper virus function (e.g., adenovirus) to trigger and/or boost AAV rep and cap gene transcription in the transfected cell. In some cases, RNA from the transfected cells provides a template for RT-PCR amplification of cDNA and the detection of novel AAVs. In cases where cells are transfected with total cellular DNAs isolated from the tissues that potentially harbor proviral AAV genomes, it is often desirable to supplement the cells with factors that promote AAV gene transcription. For example, the cells may also be infected with a helper virus, such as an Adenovirus or a Herpes Virus. In a specific embodiment, the helper functions are provided by an adenovirus. The adenovirus may be a wild-type adenovirus, and may be of human or non-human origin, preferably non-human primate (NHP) origin. Similarly adenoviruses known to infect non-human animals (e.g., chimpanzees, mouse) may also be employed in the methods of the disclosure (See, e.g., U.S. Pat. No. 6,083,716). In addition to wild-type adenoviruses, recombinant viruses or non-viral vectors (e.g., plasmids, episomes, etc.) carrying the necessary helper functions may be utilized. Such recombinant viruses are known in the art and may be prepared according to published techniques. See, e.g., U.S. Pat. No. 5,871,982 and U.S. Pat. No. 6,251,677, which describe a hybrid Ad/AAV virus. A variety of adenovirus strains are available from the American Type Culture Collection, Manassas, Va., or available by request from a variety of commercial and institutional sources. Further, the sequences of many such strains are available from a variety of databases including, e.g., PubMed and GenBank.

Cells may also be transfected with a vector (e.g., helper vector) which provides helper functions to the AAV. The vector providing helper functions may provide adenovirus functions, including, e.g., E1a, E1b, E2a, E4ORF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

In some cases, a capsid gene can be used to construct and package recombinant AAV vectors, using methods well known in the art, to determine functional characteristics associated with the novel capsid protein encoded by the gene. For example, novel isolated capsid genes can be used to construct and package recombinant AAV (rAAV) vectors comprising a reporter gene (e.g., B-Galactosidase, GFP, Luciferase, etc.). The rAAV vector can then be delivered to an animal (e.g., mouse) and the tissue targeting properties of the novel isolated capsid gene can be determined by examining the expression of the reporter gene in various tissues (e.g., heart, liver, kidneys) of the animal. Other methods for characterizing the novel isolated capsid genes are disclosed herein and still others are well known in the art.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for detecting a latent AAV in a cell. In addition, kits of the disclosure may include, instructions, a negative and/or positive control, containers, diluents and buffers for the sample, sample preparation tubes and a printed or electronic table of reference AAV sequence for sequence comparisons.

Sequences

SEQ ID NO: 1-AAV9 Capsid
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL
SEQ ID NO: 2-DNase I DNA Sequence
ATGCGGTACACAGGGCTAATGGGAACACTGCTCACCTTGGTCAACCTGCT
GCAGCTGGCTGGGACTCTGAGAATTGCAGCCTTCAACATTCGGACTTTTG
GGGAGACTAAGATGTCCAATGCTACCCTCTCTGTATACTTTGTGAAAATC
CTGAGTCGCTATGACATCGCTGTTATCCAAGAGGTCAGAGACTCCCACCT
GGTTGCTGTTGGGAAGCTCCTGGATGAACTCAATCGGGACAAACCTGACA
CCTACCGCTATGTAGTCAGTGAGCCGCTGGGCCGCAAAAGCTACAAGGAA
CAGTACCTTTTTGTGTACAGGCCTGACCAGGTGTCTATTCTGGACAGCTA
TCAATATGATGATGGCTGTGAACCCTGTGGAAATGACACCTTCAGCAGAG
AGCCAGCCATTGTTAAGTTCTTTTCCCCATACACTGAGGTCCAAGAATTT
GCGATCGTGCCCTTGCATGCAGCCCCAACAGAAGCTGTGAGTGAGATCGA
CGCCCTCTACGATGTTTACCTAGATGTCTGGCAAAAGTGGGGCCTGGAGG
ACATCATGTTCATGGGAGACTTCAATGCTGGCTGCAGCTACGTCACTTCC
TCCCAGTGGTCCTCCATTCGCCTTCGGACAAGCCCCATCTTCCAGTGGCT
GATCCCTGACAGTGCGGACACCACAGTCACATCAACACACTGTGCTTATG
ACAGGATTGTGGTTGCTGGAGCTCTGCTCCAGGCTGCTGTTGTTCCCAAC
TCGGCTGTTCCTTTTGATTTCCAAGCAGAATACGGACTTTCCAACCAGCT
GGCTGAAGCCATCAGTGACCATTACCCAGTGGAGGTGACACTCAGAAAAA
TCTGA
SEQ ID NO: 3-DNase I (GenBank ID NP_034191.3)
Protein Sequence
MRYTGLMGTLLTLVNLLQLAGTLRIAAFNIRTFGETKMSNATLSVYFVKI
LSRYDIAVIQEVRDSHLVAVGKLLDELNRDKPDTYRYVVSEPLGRKSYKE
QYLFVYRPDQVSILDSYQYDDGCEPCGNDTFSREPAIVKFFSPYTEVQEF
AIVPLHAAPTEAVSEIDALYDVYLDVWQKWGLEDIMFMGDFNAGCSYVTS
SQWSSIRLRTSPIFQWLIPDSADTTVTSTHCAYDRIVVAGALLQAAVVPN
SAVPFDFQAEYGLSNQLAEAISDHYPVEVTLRKI
SEQ ID NO: 4-Promoter Sequence
TACGGTAAATGGCCCGCCTGGCTGACCGCCCAACGACCCCGCCCATTGAC
GTCAATAATGACGTATGTTCCCATAGTAACGCCAATAGGGACTTTCCATT
GACGTCAATGGGTGGAGTATTTACGGTAAACTGCCCACTTGGCAGTACAT
CAAGTGTATCATATGCCAAGTACGCCCCCTATTGACGTCAATGACGGTAA
ATGGCCCGCCTGGCATTATGCCCAGTACATGACCTTATGGGACTTTCCTA
CTTGGCAGTACATCTACTCGAGGCCACGTTCTGCTTCACTCTCCCCATCT
CCCCCCCCTCCCCACCCCCAATTTTGTATTTATTTATTTTTTAATTATTT
TGTGCAGCGATGGGGGCGGGGGGGGGGGGGGGGGGGGCGCGCGCCAGGCG
GGGCGGGGCGGGGCGAGGGGCGGGGCGGGGCGAGGCGGAGAGGTGCGGCG
GCAGCCAATCAGAGCGGCGCGCTCCGAAAGTTTCCTTTTATGGCGAGGCG
GCGGCGGCGGCGGCCCTATAAAAAGCGAAGCGCGCGGCGGGCGGGAGCGG
GATC
SEQ ID NO: 5 AAV-DNaseI Vector Sequence
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGTAGCCATGCTCTAGGAAGATCAATTCGGTACAATTCACGCGTCGA
CATTGATTATTGACTCTGGTCGTTACATAACTTACGGTAAATGGCCCGCC
TGGCTGACCGCCCAACGACCCCGCCCATTGACGTCAATAATGACGTATGT
TCCCATAGTAACGCCAATAGGGACTTTCCATTGACGTCAATGGGTGGAGT
ATTTACGGTAAACTGCCCACTTGGCAGTACATCAAGTGTATCATATGCCA
AGTACGCCCCCTATTGACGTCAATGACGGTAAATGGCCCGCCTGGCATTA
TGCCCAGTACATGACCTTATGGGACTTTCCTACTTGGCAGTACATCTACT
CGAGGCCACGTTCTGCTTCACTCTCCCCATCTCCCCCCCCTCCCCACCCC
CAATTTTGTATTTATTTATTTTTTAATTATTTTGTGCAGCGATGGGGGCG
GGGGGGGGGGGGGGGGGGGCGCGCGCCAGGCGGGGCGGGGCGGGGCGAGG
GGCGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTA
TAAAAAGCGAAGCGCGCGGCGGGCGGGAGCGGGATCAGCCACCGCGGTGG
CGGCCCTAGAGTCGATCGAGGAACTGAAAAACCAGAAAGTTAACTGGTAA
GTTTAGTCTTTTTGTCTTTTATTTCAGGTCCCGGATCCGGTGGTGGTGCA
AATCAAAGAACTGCTCCTCAGTGGATGTTGCCTTTACTTCTAGGCCTGTA
CGGAAGTGTTACTTCTGCTCTAAAAGCTGCGGAATTGTACCCGCGGCCGA
TCCACCGGTCGCCACCATGCGGTACACAGGGCTAATGGGAACACTGCTCA
CCTTGGTCAACCTGCTGCAGCTGGCTGGGACTCTGAGAATTGCAGCCTTC
AACATTCGGACTTTTGGGGAGACTAAGATGTCCAATGCTACCCTCTCTGT
ATACTTTGTGAAAATCCTGAGTCGCTATGACATCGCTGTTATCCAAGAGG
TCAGAGACTCCCACCTGGTTGCTGTTGGGAAGCTCCTGGATGAACTCAAT
CGGGACAAACCTGACACCTACCGCTATGTAGTCAGTGAGCCGCTGGGCCG
CAAAAGCTACAAGGAACAGTACCTTTTTGTGTACAGGCCTGACCAGGTGT
CTATTCTGGACAGCTATCAATATGATGATGGCTGTGAACCCTGTGGAAAT
GACACCTTCAGCAGAGAGCCAGCCATTGTTAAGTTCTTTTCCCCATACAC
TGAGGTCCAAGAATTTGCGATCGTGCCCTTGCATGCAGCCCCAACAGAAG
CTGTGAGTGAGATCGACGCCCTCTACGATGTTTACCTAGATGTCTGGCAA
AAGTGGGGCCTGGAGGACATCATGTTCATGGGAGACTTCAATGCTGGCTG
CAGCTACGTCACTTCCTCCCAGTGGTCCTCCATTCGCCTTCGGACAAGCC
CCATCTTCCAGTGGCTGATCCCTGACAGTGCGGACACCACAGTCACATCA
ACACACTGTGCTTATGACAGGATTGTGGTTGCTGGAGCTCTGCTCCAGGC
TGCTGTTGTTCCCAACTCGGCTGTTCCTTTTGATTTCCAAGCAGAATACG
GACTTTCCAACCAGCTGGCTGAAGCCATCAGTGACCATTACCCAGTGGAG
GTGACACTCAGAAAAATCTGAAGCGGCCATCAAGCTTATCGATACCGTCG
ACTAGAGCTCGCTGATCAGCCTCGACTGTGCCTTCTAGTTGCCAGCCATC
TGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGA
GGATTGGGAAGACAATTAGGTAGATAAGTAGCATGGCGGGTTAATCATTA
ACTACAAGGAACCCCTAGTGATGGAGTTGGCCACTCCCTCTCTGCGCGCT
CGCTCGCTCACTGAGGCCGGGCGACCAAAGGTCGCCCGACGCCCGGGCTT
TGCCCGGGCGGCCTCAGTGAGCGAGCGAGCGCGCAG
SEQ ID NO: 6-mDnaseI Forward Primer
TCGCTATGACATCGCTGTTAT
SEQ ID NO: 7-mDnaseI Reverse Primer
GTCAGGTTTGTCCCGATTGAG
SEQ ID NO: 8-mGAPDH Forward Primer
TGGCAAAGTGGAGATTGTTGCC
SEQ ID NO: 9-mGAPDH Reverse Primer
AAGATGGTGATGGGCTTCCCG
SEQ ID NO: 10-Human DNase I
(GenBank ID NP_005214.2)
MRGMKLLGALLALAALLQGAVSLKIAAFNIQTFGETKMSNATLVSYIVQI
LSRYDIALVQEVRDSHLTAVGKLLDNLNQDAPDTYHYVVSEPLGRNSYKE
RYLFVYRPDQVSAVDSYYYDDGCEPCGNDTFNREPAIVRFFSRFTEVREF
AIVPLHAAPGDAVAEIDALYDVYLDVQEKWGLEDVMLMGDFNAGCSYVRP
SQWSSIRLWTSPTFQWLIPDSADTTATPTHCAYDRIVVAGMLLRGAVVPD
SALPFNFQAAYGLSDQLAQAISDHYPVEVMLK

### Examples

### Example 1. AAV-DNase I and Lung Inflammation

### Animals

AIM2 knockout and genetrap mice were generated as described in Jones et al. Proc Natl Acad Sci 107: 9771-9776 and Rathinam et al. Nat Immunol 11: 395-402. Genetrap mice were backcrossed to C57BL/6. All procedures used in this study complied with federal guidelines and were approved by the University of Massachusetts Medical School Institutional Animal Care and Use Committee.

### Influenza Virus Infection

Influenza virus A/Puerto Rico/8/1934 H1N1 (PR8) grown in chicken eggs was purchased from Charles River Laboratories (Wilmington, Mass.). Mice were first anesthetized with isofluorane and inoculated via intranasal route with 4×10⁴ pfu in 30 μL PBS.

### Measurement of Viral Load and Gene Expression

Total RNA from lung tissue was extracted using Qiazol (Qiagen) and reverse transcribed using iScript cDNA Synthesis Kit (Bio-Rad), and qPCR performed with iTaq SYBR Green Super Mix (Bio-Rad). Relative gene expression was determined using the 2ΔCt method with GAPDH as a housekeeping gene. DNase I mRNA was detected with the following primers:

mDnaseI fwd
(SEQ ID NO: 6)
5′-TCGCTATGACATCGCTGTTAT-3′,
mDnaseI rev
(SEQ ID NO: 7)
5′-GTCAGGTTTGTCCCGATTGAG-3′,
mGAPDH fwd
(SEQ ID NO: 8)
5′-TGGCAAAGTGGAGATTGTTGCC-3′,
mGAPDH rev
(SEQ ID NO: 9)
5′-AAGATGGTGATGGGCTTCCCG-3′.

Viral loads were determined by quantification of viral polymerase subunit (PA) copy number by TaqMan probes as previously described.

### Quantification of DNA in BAL

BAL was harvested using 1 mL PBS, spun at 5,000×g for 10 min and supernatants kept as cell-depleted BAL. dsDNA in BAL supernatants was quantitated by PicoGreen assay (Life Technologies)

### Flow Cytometry and Tetramer Staining

Mice were perfused with 10 mL PBS in the right ventricle. Lungs were then minced with a razor blade and strained through a 70 μM cell strainer. Cells were stained for TCRβ, CD19, B220, CD11b, CD11c, Ly6G, Ly6C, NK1.1 (eBioscience), CD45, CD4, and CD8α (BD Biosciences). For tetramer staining, cells were stained for TCRβ, CD8a, CD4, CD44 and APC-labeled tetramers (K^{b};NP_366, PA 244, PB1 703) or I-A^{b}/NP_311 (Trudeau Institute Molecular Biology Core Facility, Saranac Lake, N.Y.). Live cells were gated based on forward and side scatter, and Live/Dead Blue negative staining prior to subsequent gating. Data acquisition was performed on an LSRII (BD Biosciences) and data analyzed using FlowJo (TreeStar).

### Cytokine Quantification

Lung tissues were homogenized in 500 μL RIPA buffer, spun, and levels of IL-1β, IL-6, and TNFα in supernatants were quantified by ELISA (eBioscience).

### Generation of Recombinant Adeno-Associated Virus

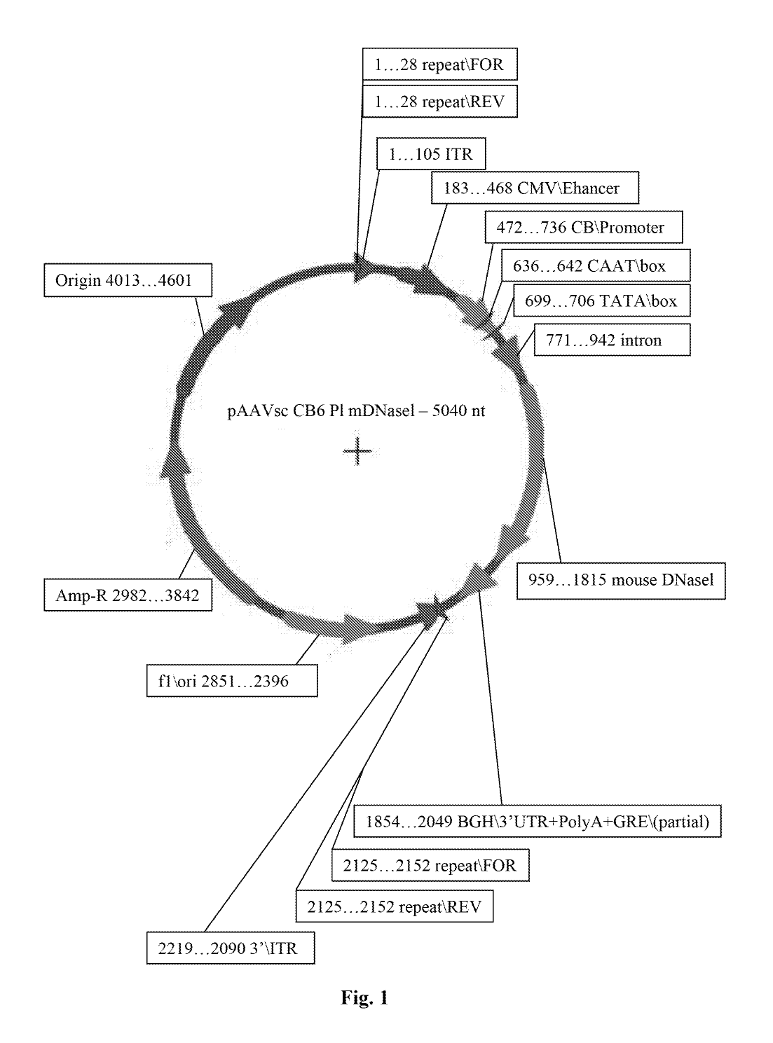

Briefly, mouse DNase I was cloned into a rAAV vector plasmid carrying a vector genome with the expression cassette driven by CMV enhanced chicken β-actin promoter and flanked by AAV2 ITRs (FIG. 1). The AAV-DNase I plasmid was co-transfected into HEK 293 cells with an AAV9 packaging plasmid and adenovirus helper plasmid. The recombinant virus was purified by standard CsCl gradient sedimentation method and desalted by dialysis. Mice were first anesthetized with isofluorane and inoculated via intranasal route with 10¹⁰ pfu in 30 μL PBS at least two weeks prior to experiments.

### Statistical Analysis

Data were analyzed using the two-tailed Student's t test comparing means between groups. Kaplan-Meier survival curves were analyzed by Mantel-Cox log-rank test. A p-value <0.05 was considered statistically significant. Graphing and statistical analyses were done using GraphPad Prism.

### Accumulated DNA in the Lung Microenvironment During IAV Infection is Protective.

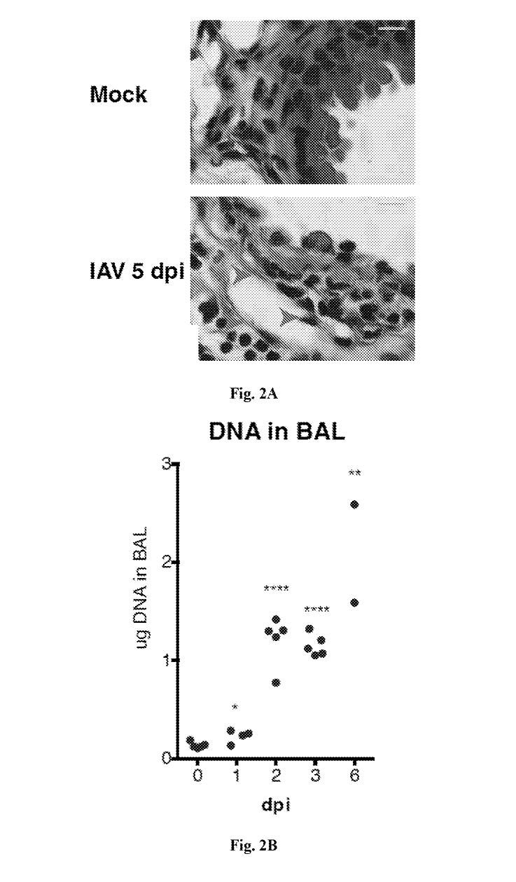

Extracellular DNA extruded from necrotic cells was noticed primarily localized to the bronchi of the lungs of Influenza virus infected mice (FIG. 2A). One source of this DNA is necrotic bronchiolar epithelial cells or neutrophil extracellular traps (NETs). The levels of extracellular DNA released was formally quantified within tissues by obtaining bronchiolar lavage (BAL) fluid that was depleted of host cells. Uninfected mice had low, albeit detectable quantities of dsDNA (FIG. 2B). The amount of DNA in the BAL increased significantly over uninfected levels as early as 1 dpi and continued to increase in step with the spread of infection at 2, 3, and 6 dpi (FIG. 2B).

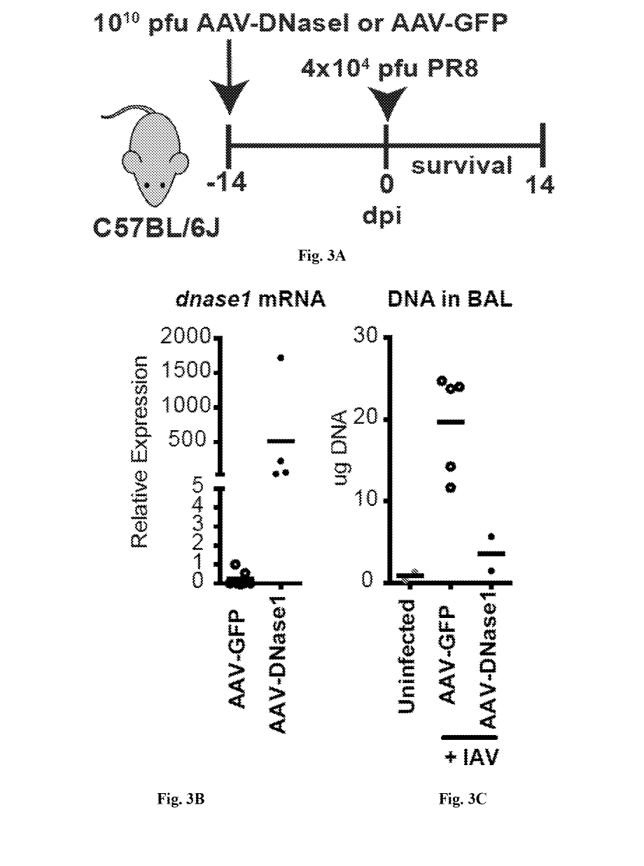

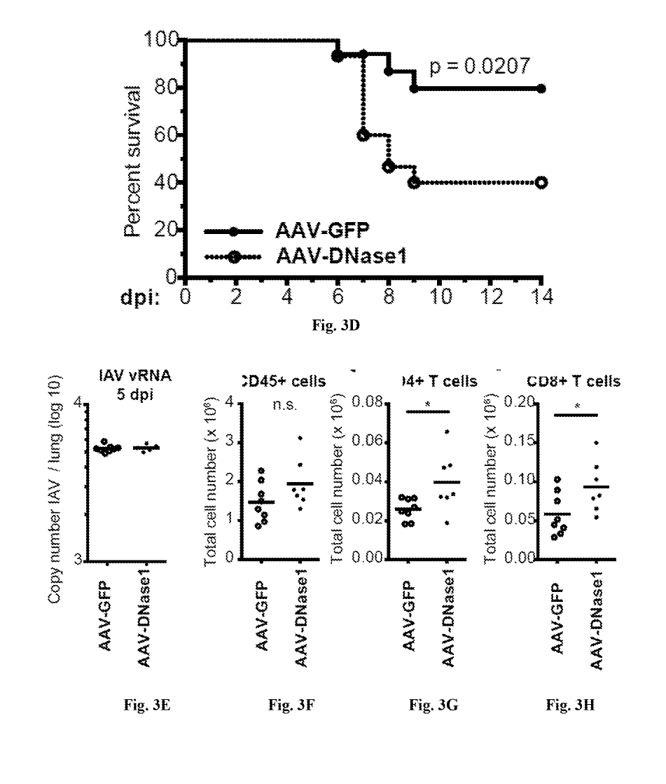

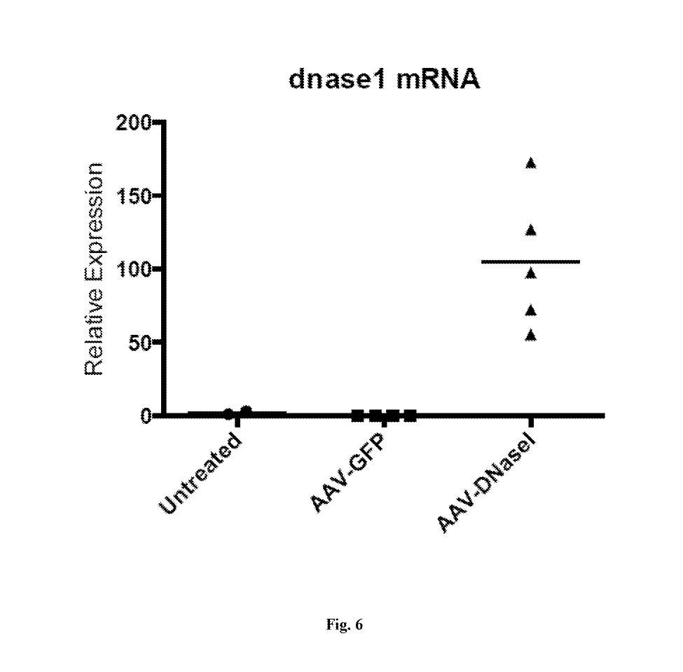

To test the role of DNA on the quality or duration of the immune response, a recombinant adeno-associated virus (AAV) to ectopically express mouse DNase I in the lungs of C57BL/6 mice was generated (FIG. 3A). DNase I is a secreted protein, normally absent from the lung with respect to its enzymatic activity and expression (FIG. 3B). Intranasal infection with AAV-DNase I resulted in robust and stable expression of the transgene in the lung 2 weeks after transduction (FIG. 3B), and at least out to 3 months (FIG. 6). The levels of DNA in the BAL fluid of IAV infected mice were significantly reduced in mice treated with AAV-DNase I compared to GFP expressing control virus, confirming the product of the DNase I transgene was secreted and enzymatically active (FIG. 3C).

AAV-DNase I-mediated ablation of DNA in the lung microenvironment during IAV infection impacted the susceptibility of mice to lethality was studied. Mice treated with AAV-DNase I showed a significant decrease in survival (40%) compared to controls (79.6%) out to 14 dpi following challenge with an LD25 of PR8 (FIG. 3D). It was next tested if the increased mortality of the AAV-DNase I treated mice was due to unchecked viral replication. Measurement of PR8 viral RNA copy number in total RNA from the lungs of AAV-GFP and AAV-DNase I treated mice at day 5 pi revealed similar viral loads (FIG. 3E). Similar observations were made using plaque assay to measure infectious virus production. FACS analysis of infiltrating leukocyte populations in the lungs of AAV-DNase I revealed a significant increase in the number of CD4⁺ and CD8⁺ T cells compared to controls at 5 dpi (FIGS. 3F-H). Combined, these data suggest that DNA present within the lung microenvironment during IAV infection is protective, and may serve to limit the number of infiltrating T cells able to drive the development immune pathology.

### AIM2 Regulates Inflammatory Responses in the Lung During IAV Infection.

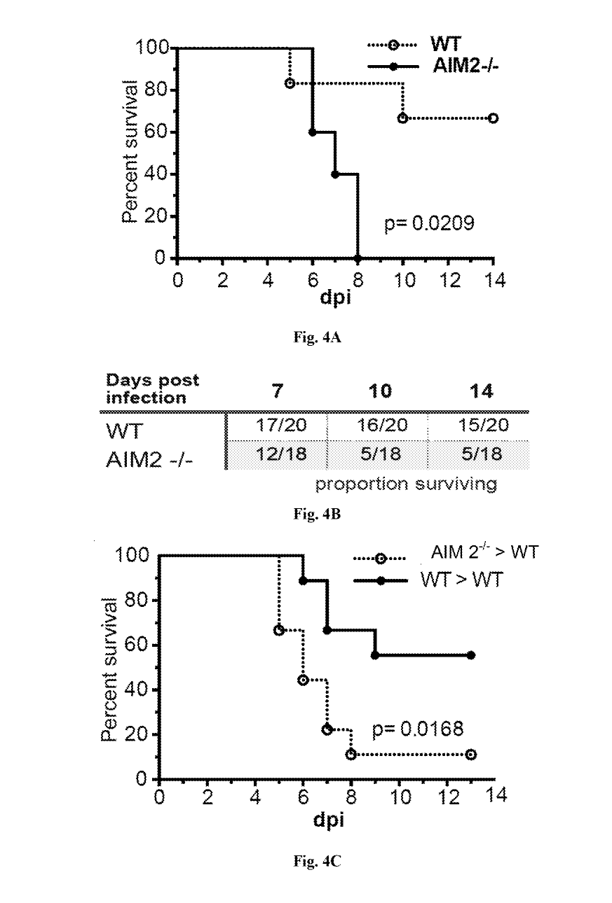

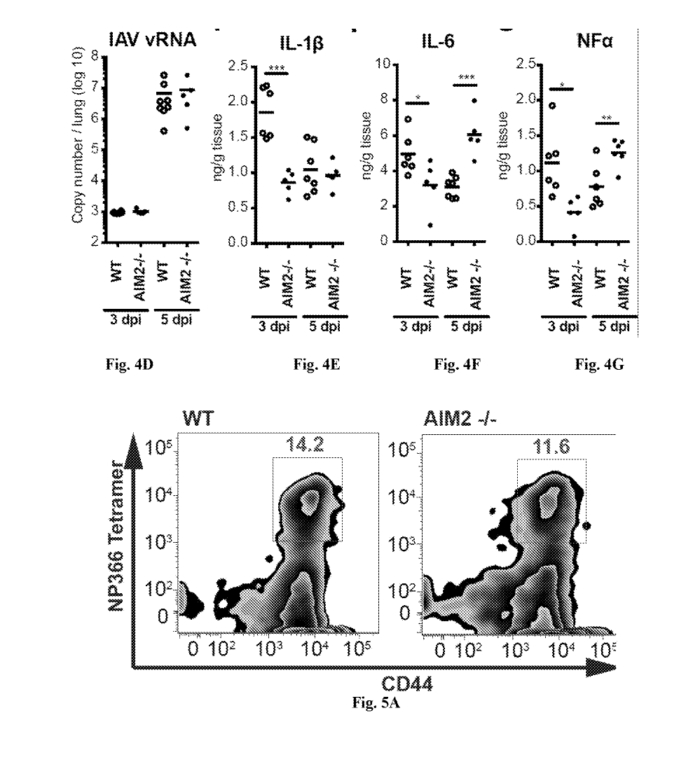

Treatment with AAV-DNase I was tested in mice lacking Absent in Melanoma (AIM)-2, a well described cytosolic DNA receptor required for activation of the inflammasome complex in response to dsDNA. Previous studies have identified roles for inflammasomes in IAV infection. IAV infection has been shown to regulate IL-1β via the NLRP3 inflammasome. Each component of the NLRP3 inflammasome, including ASC and caspase-1, have been shown to be required to drive the inflammatory response and leukocyte recruitment during IAV infection. To test the contribution of AIM2, during IAV infection, mice lacking AIM2 were infected with a lethal IAV challenge. While 75% of WT mice survived IAV infection, only 28% of AIM2^{-/-} mice were surviving at 14 dpi (FIG. 4A and FIG. 4B). Bone marrow transplants were performed to elucidate whether expression of AIM2 in hematopoietic cells is required for protection from lethal IAV infection. Here, WT mice engrafted with Aim2^{-/-} bone marrow were significantly more susceptible to IAV infection compared to those receiving WT bone marrow (FIG. 4C). There was no difference in IAV viral RNA copy number in the lungs of WT and Aim2^{-/-} mice at day 3 and day 5 dpi (FIG. 4D). Similar results obtained by plaque assay further confirmed that AIM2, while required for protection during lethal IAV challenge, is dispensable for controlling IAV replication.

It was next assessed whether the susceptibility of Aim2^{-/-} mice correlated with a defect in IL-1β production. qPCR analysis of IL-1β, IL-1α and IL-1R antagonist (IL-1Ra) mRNA showed no defect in their induction between Aim2^{-/-} and WT mice at 3 and 5 dpi. However, the levels of IL-1β protein in lung homogenates were significantly lower at 3 dpi in AIM2-deficient mice. These levels increased by 5 dpi to match those of WT mice (FIG. 4E). Similar to the effects observed early for IL-1β levels, it was found that IL-6 and TNFα were also decreased in lung homogenates of Aim2^{-/-} mice at 3 dpi compared to WT controls (FIGS. 4F and 4G). However, by 5 dpi, the levels of both TNFα and IL-6 were significantly elevated in Aim2^{-/-} mice (FIGS. 4F and 4G). The early effects of AIM2-deficiency on these cytokines may result from decreased IL-1β production and IL-1R dependent induction of these cytokines, or may reflect a greater number of leukocytes able to produce cytokines in the lungs of Aim2^{-/-} mice.

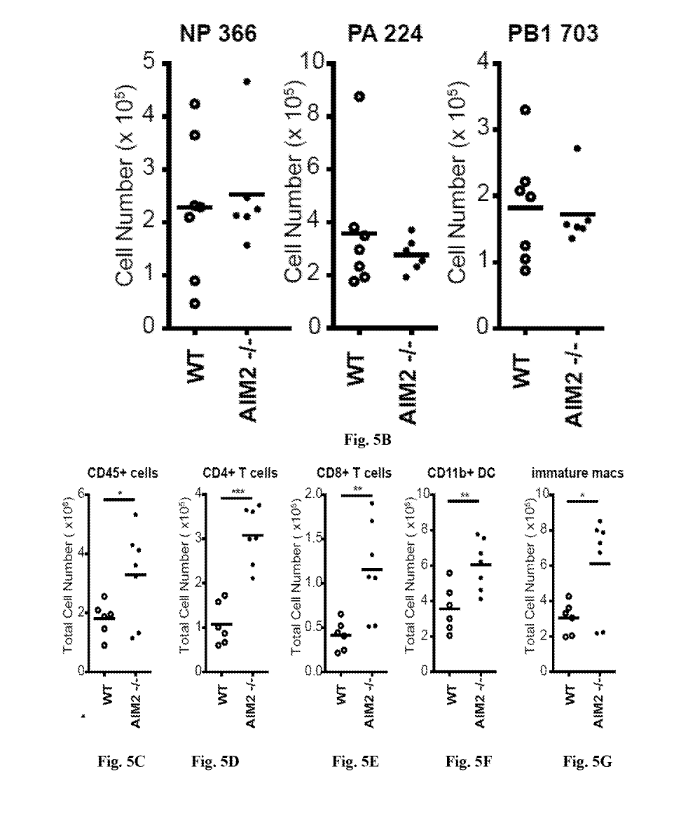

IL-1R signaling during IAV infection is required for eliciting a protective flu-specific T cell response. AIM2 is required for IL-1β production upon sensing DNA in the cytosol, thus whether AIM2 deficiency impaired the formation of flu-specific T cells was investigated. After infection with a sublethal dose of PR8, tetramer staining was performed using single-cell suspensions from the lung and spleen with four class I and one class II IAV epitopes to determine the frequency and number of flu-specific CD8⁺ and CD4⁺ T cells, respectively. No significant difference in the frequency (FIG. 5A) of NP_366-tetramer⁺ CD8⁺ T cells in the lungs at 9 dpi was found. Similarly, the numbers of CD8⁺ T cells positive for tetramers bearing three different IAV epitopes (NP_366, PA 224, PB1 703) were unaffected by AIM2-deficiency (FIG. 5B). Similar results were found in both the lung and spleen for all class I and class II epitopes tested. These data indicate that AIM2 plays a protective role during IAV infection and that the protective effect is unlikely due to the limited effects of AIM2 on early IL-1β production or the formation of flu-specific T cell responses all of which are unaffected by AIM2-deficiency.

Flow cytometric analysis was used to examine whether the absence of AIM2 altered leukocyte recruitment to the lungs of IAV infected mice. This analysis revealed a significant increase in CD45⁺ leukocytes in the lungs of Aim2^{-/-} mice compared to WT controls in single-cell suspensions prepared from total lung at 5 dpi (FIG. 5C). Looking at individual populations of immune cells within the CD45⁺ population, it was found that the increased number of leukocytes in the lungs of Aim2^{-/-} mice was due to an increase in the total numbers of CD4⁺ and CD8⁺ T cells, CD11b⁺ dendritic cells, and immature macrophages (FIG. 5D-FIG. 5G). No significant difference in either the frequency or number of neutrophils, B cells, monocytes, or NK cells was found.

Overall, the studies demonstrate that host-derived DNA accumulates in lung tissue damaged by IAV infection and has the capacity to modulate the intensity of the host response. This extracellular DNA in turn engages the AIM2 pathway to curb further immune-mediated tissue damage. Using a mouse influenza model, levels of host-derived DNA in the lung continue to rise as the infection spreads throughout the tissue, resulting in necrosis of the infected cells.

Mice lacking AIM2, a cytosolic sensor primarily known for its role in controlling inflammasome activation in response to cytosolic DNA, succumbed to infection. Although AIM2 impacted the magnitude of the IAV induced IL-1β response in vivo, this response was indistinguishable from that of wild type mice at later time points. Consistent with a largely intact IL-1β response, Aim2^{-/-} mice were able to mount normal flu-specific adaptive responses, which are known to lie downstream of IL-1R signaling. AIM2-deficient mice had similar viral loads to those seen in wild type mice, indicating that the effect of AIM2 in vivo was also not due to a failure to curb viral replication. Numbers of infiltrating leukocytes were increased however in the lungs of Aim2^{-/-} mice. Moreover, levels of TNFα and IL-6, albeit slightly decreased early during infection, were also produced at levels greater than WT mice at 5 dpi likely due to the increased number of leukocytes in the lungs of Aim2^{-/-} mice. Consistent with the findings that a DNA sensor is able to regulate inflammation and morbidity during infection with an RNA virus, it was also found that ablation of DNA in the lung microenvironment by ectopic DNase I expression yielded a similar effect to that observed in Aim2^{-/-} mice. Collectively, these findings suggest that accumulation of DNA in tissues damaged by infection may provide a mechanism for alerting the immune system to the extent of tissue damage and functions as a signal to limit excessive immune pathology.

### Example 2. Inhibition of Biofilm Formation by AAV-DNase I

DNase I has been used primarily for the treatment of CF. However, there are number of other conditions and situations where long-term DNAse I expression (e.g., AAV-DNase I) could be therapeutically beneficial, for example removal and prevention of biofilms. Biofilms are formed when bacteria attach to surfaces and form structured multicellular communities. These biofilm communities create extracellular matrices as a means to provide shelter from chemical, biological, and physical damage. A major component of biofilms contributing to their structural integrity is extracellular DNA. DNase I is effective in preventing the formation and disrupting preexisting biofilm formation by a wide variety of gram-negative and gram-positive bacteria, as well as fungi. The formation of biofilms are implicated in the development of persistent disease, one example being Gardneralla vaginalis biofilms in bacterial vaginosis. Treatment of preexisting G. vaginalis biofilms in the vagina of female mice with recombinant DNase I rendered the bacteria sensitive to antibiotic killing and prevented new biofilms from colonizing. DNase I has also been shown to similarly increase the effectiveness in antibiotic therapies for a number of bacterial species.

In addition to host tissues, prosthetic and medical implants are also commonly colonized by biofilms. It is estimated that contamination of these devices by biofilms leading to clinical symptoms occurs anywhere from 1% to 6% of patients receiving prosthetic grafts. These cases have catastrophic consequences including surgical procedures to remove the contaminated device or sepsis leading to mortality. Biofilms formed on prosthetic grafts are extremely resistant to antibiotic treatment. Taken together, the use of AAV-DNase I may provide a useful therapeutic tool for the prevention of biofilm formation in patients with prosthetic grafts or those suffering from persistent infection from these microorganisms, as well as increasing the efficacy of antibiotics in these cases.

### Example 3. AAV-DNase I and Autoimmune Disease

The accumulation of self-nucleic acids, including DNA, is linked to the development and progression of autoimmune disease. Self-nucleic acids (e.g., DNA) is sensed by the receptors of the innate immune system leading to the activation of signaling pathways culminating in the production of inflammatory cytokines, chemokines, interferons (IFN), etc. DNA and RNA released from necrotic cells or misplaced intracellular nucleic acids may be recognized by innate immune receptors. Continuous activation of these inflammatory signaling pathways may eventually lead to the loss of self-tolerance by the immune system, causing the host's own cells become targeted by the host's adaptive immune response.

Systemic lupus erythematosus (SLE) patients show high expression of genes induced by type I IFN which is predominately triggered by the sensing of nucleic acids by the innate immune system. Decreased DNase I activity positively correlates with disease severity in SLE patients. Mice deficient for the dnase1 gene develop an SLE-like disease, including the presence of anti-nuclear antibodies (ANA) and development of glomerulonephritis. In NZB/NZW F1 mice, which spontaneously develop SLE, repeated administration recombinant DNase I after the onset of disease increases survival and improves kidney damage.

Mice deficient for the lysosomal dnase2a are embryonic lethal due to type I IFN mediated anemia. DNA from the nuclei of erythroid progenitors accumulates in fetal macrophages, driving the production of type I IFN. This lethality can be rescued by the deletion of the type I IFN receptor. However, these mice go on to develop severe polyarthritis. TREX1 (aka DNase3), a DNase localized to the endoplasmic reticulum, has been shown to be critical for the removal of intracellular DNA derived from endogenous retroelements. Trex1-deficient mice develop an autoimmune disease with similar symptoms as SLE. Supporting these findings, mutations in the human trex1 gene cause Aicardi-Goutieres Syndrome (AGS) and chilblain lupus.

Clearance of unwanted host DNA and preventing its accumulation are important processes for preventing autoimmunity. Current therapeutics for autoimmune disease are targeted at treating the symptoms rather than the proximal events leading to the development of inflammation and eventual loss of tolerance. The AAV-DNase1 vector may prove a useful therapeutic for a number of autoimmune diseases by providing a continuous source of enzyme for removing DNA, and preventing its sensing by the innate immune system. The location of transgene expression by AAV vectors is largely influenced by the tropism of the serotype used for packaging. Packaging of the dnase1 transgene into serotypes that more efficiently target the affected tissue could further increase its efficacy.

What is claimed is:

1. A recombinant AAV (rAAV) comprising: a capsid protein having a sequence as set forth in SEQ ID NO: 1 and a nucleic acid comprising a promoter operably linked to a transgene, wherein the transgene encodes a nuclease.

2. The rAAV of claim 1, wherein the nuclease is DNase I.

3. The rAAV of claim 2, wherein the DNase I is human DNase I or mouse DNase I.

4. The rAAV of any one of claims 1 to 3, wherein the transgene comprises a sequence as set forth in SEQ ID NO: 2 or encodes a protein as set forth in SEQ ID NO: 3 or 10.

5. The rAAV of any one of claims 1 to 4, wherein the nucleic acid further comprises two AAV inverted terminal repeats (ITRs), wherein the ITRs flank the transgene.

6. The rAAV of claim 5, wherein the AAV ITRs are ITRs of one or more serotypes selected from: AAV2, AAV3, AAV4, AAV5, and AAV6.

7. The rAAV of any one of claims 1 to 6, wherein the promoter comprises a sequence as set forth in SEQ ID NO: 4.

8. A composition comprising the rAAV of any one of claims 1 to 7 and a pharmaceutically acceptable carrier.

9. An isolated nucleic acid having the sequence as set forth in SEQ ID NO: 5.

10. A vector comprising the isolated nucleic acid of claim 9.

11. A host cell comprising the nucleic acid of claim 9 or the vector of claim 10.

12. The host cell of claim 11, wherein the host cell is a human cell or bacterial cell.

13. A method for delivering a transgene to lung tissue, the method comprising: administering to lung tissue of a subject an effective amount of rAAV, wherein the rAAV comprises (i) a capsid protein and (ii) a nucleic acid comprising a promoter operably linked to a transgene, wherein the transgene encodes a nuclease.

14. The method of claim 13, wherein the nuclease is DNase I.

15. The method of claim 14, wherein the DNase I is human DNase I or mouse DNase I.

16. The method of any one of claims 13 to 15, wherein the transgene comprises a sequence as set forth in SEQ ID NO: 2 or encodes a protein as set forth in SEQ ID NO: 3 or 10.

17. The method of any one of claims 13 to 16, wherein the capsid protein is selected from the group consisting of: AAV2, AAV3, AAV4, AAV5, AAV6, AAV7, AAV8, or AAV9 capsid protein.

18. The method of claim 17, wherein the capsid protein comprises the sequence set forth in SEQ ID NO: 1.

19. The method of any one of claims 13 to 18, wherein the nucleic acid further comprises two AAV inverted terminal repeats (ITRs), wherein the ITRs flank the transgene.

20. The method of claim 19, wherein the AAV ITRs are ITRs of one or more serotypes selected from: AAV2, AAV3, AAV4, AAV5, and AAV6.

21. The method of any one of claims 13 to 20, wherein the promoter comprises a sequence as set forth in SEQ ID NO: 4.

22. The method of any one of claims 13 to 21, wherein the administration is respiratory administration.

23. The method of claim 22, wherein the respiratory administration comprises intranasal or intratracheal administration.

24. The method of claim 22 or 23, wherein the respiratory administration comprises administration of aerosolized particles comprising the rAAV.

25. The method of any one of claims 22 to 24, wherein the respiratory administration is a self-administration.

26. The method of claim 25, wherein the respiratory administration is a self-administration by inhalation.

27. A method for treating a lung-associated disease, the method comprising: administering to a subject having or suspected of having a lung-associated disease an effective amount of rAAV, wherein the rAAV comprises (i) a capsid protein and (ii) a nucleic acid comprising a promoter operably linked to a transgene, wherein the transgene encodes a nuclease.

28. The method of claim 27, wherein the treatment comprising administering a dose of the rAAV to the subject no more than once within a calendar day.

29. The method of claim 28, wherein the treatment comprising administering a dose of the rAAV to the subject no more than once within a calendar week.

30. The method of claim 29, wherein the treatment comprising administering a dose of the rAAV to the subject no more than once within a calendar month.

31. The method of any one of claims 27 to 30, wherein the nuclease is DNase I.

32. The method of claim 31, wherein the DNase I is human DNase I or mouse DNase I.

33. The method of any one of claims 27 to 32, wherein the transgene comprises a sequence as set forth in SEQ ID NO: 2 or encodes a protein as set forth in SEQ ID NO: 3 or 10.

34. The method of any one of claims 27 to 33, wherein the capsid protein is selected from the group consisting of: AAV2, AAV3, AAV4, AAV5, AAV6, AAV7, AAV8, or AAV9 capsid protein.

35. The method of claim 34, wherein the capsid protein comprises the sequence set forth in SEQ ID NO: 1.

36. The method of any one of claims 27 to 35, wherein the nucleic acid further comprises two AAV inverted terminal repeats (ITRs), wherein the ITRs flank the transgene.

37. The method of claim 36, wherein the AAV ITRs are ITRs of one or more serotypes selected from: AAV2, AAV3, AAV4, AAV5, and AAV6.

38. The method of any one of claims 27 to 37, wherein the promoter comprises a sequence as set forth in SEQ ID NO: 4.

39. The method of any one of claims 27 to 38, wherein the administration is respiratory administration.

40. The method of any one of claims 27 to 39, wherein the lung-associated disease is selected from the group consisting of: cystic fibrosis, asthma, chronic obstructive pulmonary disease (COPD), emphysema, bronchitis, pneumonia, tuberculosis, influenza, pulmonary edema, and acute respiratory distress syndrome (ARDS).

41. A method for inhibiting biofilm formation on a surface, the method comprising: administering to a surface an effective amount of rAAV, wherein the rAAV comprises (i) a capsid protein and (ii) a nucleic acid comprising a promoter operably linked to a transgene, wherein the transgene encodes a nuclease.

42. The method of claim 41, wherein the surface is a hard tissue or a soft tissue.

43. The method of claim 41, wherein the surface is a non-biological surface.

44. The method of any one of claims 41 to 43, wherein the surface is in vivo.

45. The method of claim 41, wherein the surface is a surface of a biomedical implant.

46. A method for treating an autoimmune disease, the method comprising: administering to a subject having or suspected of having a lung-associated disease an effective amount of rAAV, wherein the rAAV comprises (i) a capsid protein and (ii) a nucleic acid comprising a promoter operably linked to a transgene, wherein the transgene encodes a nuclease.

47. The method of claim 46, wherein the autoimmune disease is systemic lupus erythematosus (SLE), polyarthritis, Aicardi-Goutieres Syndrome (AGS), or chilblain lupus.
